(19) United States
(12) Patent Application Publication
(10) Pub. No.: US 2021/0132028 A1
(43) Pub. Date: May 6, 2021

(54) SYSTEMS AND METHODS FOR MONITORING FIELD CONDITIONS

(71) Applicant: CNH Industrial Canada, Ltd., Saskatoon (CA)

(72) Inventors: Christopher A. Foster, Mohnton, PA (US); James W. Henry, Saskatoon (CA); John H. Posselius, Ephrata, PA (US)

(73) Assignee: CNH Industrial Canada, Ltd.

(21) Appl. No.: 16/674,641

(22) Filed: Nov. 5, 2019

(51) Int. Cl. G01N 33/24; A01B 79/00

(52) U.S. Cl. CPC G01N 33/24; G01N 2033/245; A01B 79/005

(57) ABSTRACT

A system for monitoring field conditions of a field includes a sensor supported on an agricultural implement, the sensor having a field of view directed towards an aft portion of the field disposed rearward of the agricultural implement relative to a direction of travel of the agricultural implement. The sensor generates data indicative of a field condition associated with the aft portion of the field. An actuator actuates the sensor back and forth relative to the agricultural implement along a sensor movement path. A controller receives data from the sensor indicative of the field condition as the actuator actuates the sensor such that the field of view of the sensor is oscillated across the aft portion of the field while the agricultural implement is being moved across the field. The controller monitors the field condition based at least in part on the data received from the sensor.

### FIELD OF THE INVENTION

The present disclosure relates generally to systems and methods for monitoring field conditions and, more particularly to systems for monitoring field conditions as an agricultural implement moves across a field.

### BACKGROUND OF THE INVENTION

It is well known that, to attain the best agricultural performance from a field, a farmer must cultivate the soil, typically through a tillage operation. Tillage implements typically include one or more ground engaging tools configured to engage the soil as the implement is moved across the field. Such ground engaging tool(s) loosen and/or otherwise agitate the soil to prepare the field for subsequent agricultural operations, such as planting operations. The field conditions after a tillage operation, such as surface roughness and residue coverage, impact subsequent farming operations within the field. In this regard, sensor systems have been developed that allow field conditions to be detected along a portion of the field behind the tillage implement during the tillage operation.

However, conventional sensor systems typically include a fixed sensor having a limited field of view. As such, field conditions may only be captured for a small portion of the field behind the implement. Such issue can potentially be addressed with the use of multiple fixed sensors. However, multi-sensor system arrangements are often prohibitively expensive.

Accordingly, improved systems and methods for monitoring field conditions as an agricultural implement is moved across a field would be welcomed in the technology.

### BRIEF DESCRIPTION OF THE INVENTION

Aspects and advantages of the invention will be set forth in part in the following description, or may be obvious from the description, or may be learned through practice of the invention.

In one aspect, the present subject matter is directed to a system for monitoring field conditions of a field. The system includes a sensor, an actuator, and a controller. The sensor is supported on an agricultural implement such that the sensor has a field of view directed towards an aft portion of the field disposed rearward of the agricultural implement relative to a direction of travel of the agricultural implement. The sensor is configured to generate data indicative of a field condition associated with the aft portion of the field. The actuator is configured to actuate the sensor back and forth relative to an adjacent portion of the agricultural implement along a sensor movement path. The controller is configured to receive data from the sensor indicative of the field condition as the actuator actuates the sensor back and forth along the sensor movement path such that the field of view of the sensor is oscillated across the aft portion of the field while the agricultural implement is being moved across the field. The controller is further configured to monitor the field condition based at least in part on the data received from the sensor.

In further aspect, the present subject matter is directed to another system for monitoring field conditions of a field. The system includes a sensor supported on an agricultural implement such that the sensor has a field of view directed towards the field, where the sensor is configured to generate data indicative of a field condition associated with the field. The system further includes an actuator configured to actuate the sensor back and forth relative to an adjacent portion of the agricultural implement along a sensor movement path. The system additionally includes a controller configured to determine an area-of-interest within the field. The controller being further configured to control an operation of the actuator to actuate the sensor along the sensor movement path such that the field of view is directed towards the area-of-interest within the field. The controller being additionally configured to monitor the field condition associated with the area-of-interest based at least in part on the data received from the sensor.

In another aspect, the present subject matter is directed to yet another system for monitoring field conditions of a field. The system includes a sensor supported on an agricultural implement, where the sensor has a field of view directed towards a portion of the field. The sensor is configured to generate data indicative of afield condition associated with the portion of the field. The system further includes an actuator configured to linearly actuate the sensor back and forth relative to an adjacent portion of the agricultural implement along a linear movement path. The system additionally includes a controller configured to receive data from the sensor indicative of the field condition as the actuator linearly actuates the sensor back and forth along the linear movement path such that the field of view of the sensor is oscillated across the portion of the field while the agricultural implement is being moved across the field. The controller is further configured to monitor the field condition based at least in part on the data received from the sensor.

In a further aspect, the present subject matter is directed to a method for monitoring field conditions of a field. The method includes receiving, with a computing device, data from a sensor indicative of a field condition as an actuator actuates the sensor back and forth along a sensor movement path such that a field of view of the sensor is oscillated across a portion of the field disposed relative to an agricultural implement while the agricultural implement is being moved across the field. The method further includes monitoring, with the computing device, the field condition based at least in part on the data received from the sensor. The method additionally includes performing, with the computing device, a control action based on the monitored field condition.

In an additional aspect, the present subject matter is directed to another method for monitoring field conditions of a field. The method includes receiving, with a computing device, an input associated with determining an area-of-interest within a field while an agricultural implement is being moved across the field. The method further includes controlling, with the computing device, an operation of an actuator to actuate a sensor along a sensor movement path such that a field of view of the sensor is directed towards the area-of-interest, the sensor being configured to generate data indicative of a field condition within the area-of-interest. Additionally, the method includes monitoring, with the computing device, a field condition associated with the area-of-interest based at least in part on data received from the sensor.

These and other features, aspects and advantages of the present invention will become better understood with reference to the following description and appended claims. The accompanying drawings, which are incorporated in and constitute a part of this specification, illustrate embodiments of the invention and, together with the description, serve to explain the principles of the invention.

### BRIEF DESCRIPTION OF THE DRAWINGS

A full and enabling disclosure of the present invention, including the best mode thereof, directed to one of ordinary skill in the art, is set forth in the specification, which makes reference to the appended figures, in which:

FIG. 1 illustrates a perspective view of one embodiment of an agricultural implement coupled to a work vehicle in accordance with aspects of the present subject matter;

FIG. 2 illustrates another perspective view the agricultural implement shown in FIG. 1 in accordance with aspects of the present subject matter;

FIG. 3 illustrates a schematic, top down view of one embodiment of a system for monitoring field conditions provided in operative association with the agricultural implement and the work vehicle shown in FIGS. 1 and 2 in accordance with aspects of the present subject matter;

FIG. 4 illustrates one embodiment of a sensor movement path of a sensing assembly in accordance with aspects of the present subject matter:

FIG. 5 illustrates another embodiment of a sensor movement path of a sensing assembly in accordance with aspects of the present subject matter;

FIG. 6 illustrates an example view of an aft end of the implement shown in FIG. 3 and an adjacent portion of a field in accordance with aspects of the present subject matter;

FIG. 7 illustrates a schematic view of a system for monitoring field conditions in accordance with aspects of the present subject matter;

FIG. 8 illustrates another example view of an aft end of the implement shown in FIG. 3 and an adjacent portion of a field, particularly illustrating an area-of-interest within the field in accordance with aspects of the present subject matter:

FIG. 9 illustrates a flow diagram of one embodiment of a method for monitoring field conditions in accordance with aspects of the present subject matter; and

FIG. 10 illustrates a flow diagram of another embodiment of a method for monitoring field conditions in accordance with aspects of the present subject matter.

Repeat use of reference characters in the present specification and drawings is intended to represent the same or analogous features or elements of the present technology.

### DETAILED DESCRIPTION OF THE INVENTION

Reference now will be made in detail to embodiments of the invention, one or more examples of which are illustrated in the drawings. Each example is provided by way of explanation of the invention, not limitation of the invention. In fact, it will be apparent to those skilled in the art that various modifications and variations can be made in the present invention without departing from the scope or spirit of the invention. For instance, features illustrated or described as part of one embodiment can be used with another embodiment to yield a still further embodiment. Thus, it is intended that the present invention covers such modifications and variations as come within the scope of the appended claims and their equivalents.

In general, the present subject matter is directed to systems and methods for monitoring field conditions of a field as an agricultural implement moves across the field. Specifically, in several embodiments, a computing device or controller of the disclosed system may be configured to monitor one or more field conditions based on data received from a sensor provided in operative association with an agricultural implement performing an operation within the field. The sensor may have a field of view directed towards a portion of the field such that the sensor generates data indicative of the monitored field condition(s) associated with such portion of the field. Additionally, in accordance with aspects of the present subject matter, the sensor may be configured to be moved or actuated back and forth along a sensor movement path such that the field of view of the sensor is oscillated across an adjacent portion of the field while the agricultural implement is being used to perform an operation within the field. As such, the sensor may capture data associated with the monitored field condition(s) across a larger area of the field than if the sensor were fixed in position. In some embodiments, the sensor movement path may be linear, such that the sensor is linearly oscillated back and forth along the linear movement path. Additionally or alternatively, in some embodiments, the sensor movement path may be arced or curved such that the sensor is pivotably oscillated back and forth along the arced movement path.

Moreover, in accordance with aspects of the present subject matter, the system controller may be configured to determine an area-of-interest within the field. For instance, in one embodiment, the controller may monitor the field condition data received from the sensor to determine an area-of-interest within the field. In other embodiments, the controller may receive an indication of a desired area-of-interest within the field from an operator. In further embodiments, the controller may monitor additional or supplemental data from one or more secondary sensors configured to detect parameters indicative of operating parameters of the implement, such as vibrations, levelness, etc., and/or other field conditions, such as moisture content, etc. Upon the determination of an area-of-interest within the field, the sensor may be moved along its associated sensor movement path such that the field of view of the sensor is directed towards the area-of-interest, thereby allowing the controller to specifically monitor the field condition(s) within the area-of-interest. In one embodiment, the controller may be configured to adjust the operation of the implement based on the determined condition(s) within the area-of-interest.

Additionally, in accordance with aspects of the present subject matter, the controller may also be configured to generate a field condition map for the field based at least in part on the data received from the sensor. More particularly, the data received from the sensor may be geo-referenced such that an estimated field condition(s) may be determined at each location within the field. However, in certain instances, the data received from the sensor will only correspond to a portion of the field as the sensor is being oscillated back and forth along its associated sensor movement path. Thus, in such instances, the controller may be configured to estimate the associated field condition(s) of one or more portions of the field outside of the field of view of the sensor based on the data received from the sensor to “fill-out” the field condition map. The field condition map may then be used, for example, to control the operation of the implement performing the current field operation or an implement performing a subsequent field operation.

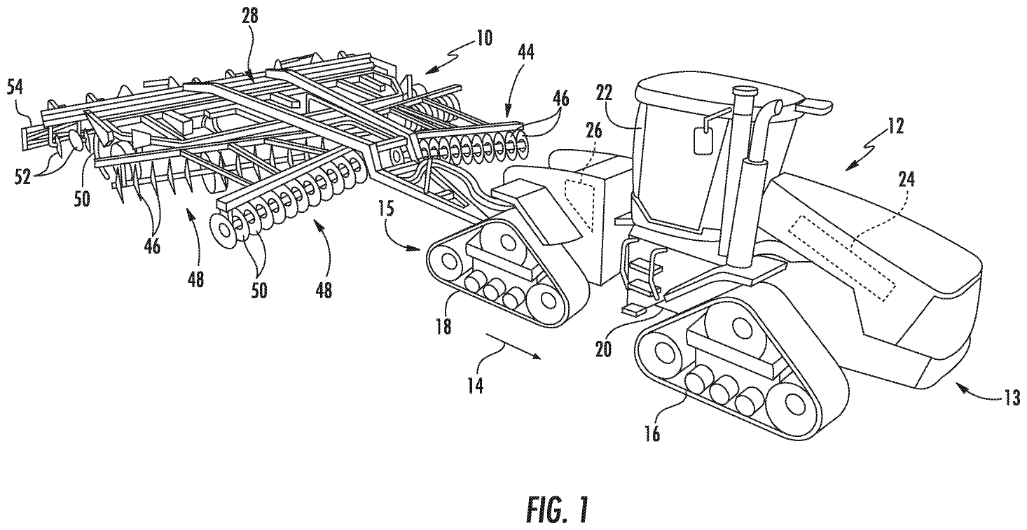

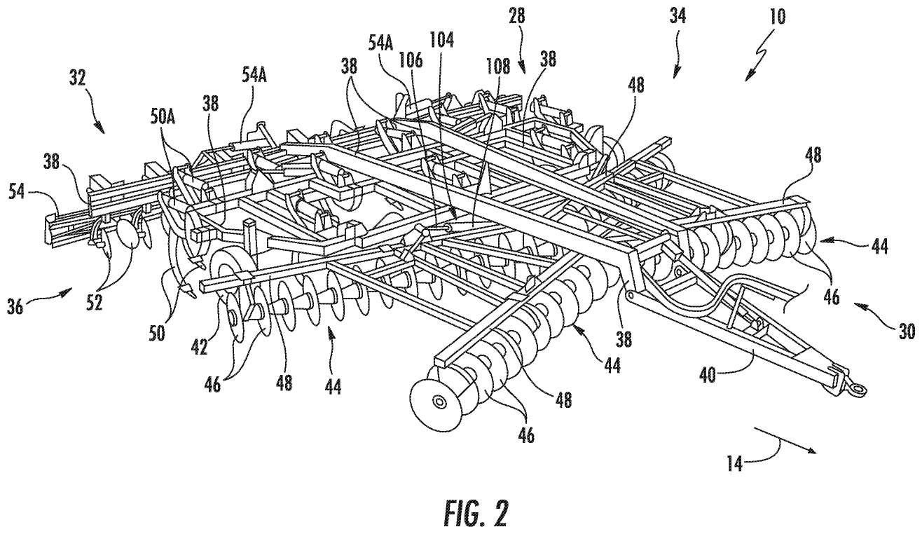

Referring now to the drawings, FIGS. 1 and 2 illustrate differing perspective views of one embodiment of an agricultural implement 10 in accordance with aspects of the present subject matter. Specifically, FIG. 1 illustrates a perspective view of the agricultural implement 10 coupled to a work vehicle 12. Additionally, FIG. 2 illustrates a perspective view of the implement 10, particularly illustrating various components of the implement 10.

In general, the implement 10 may be configured to be towed across a field in a direction of travel (e.g., as indicated by arrow 14 in FIG. 1) by the work vehicle 12. As shown, the implement 10 may be configured as a tillage implement, and the work vehicle 12 may be configured as an agricultural tractor. However, in other embodiments, the implement 10 may be configured as any other suitable type of implement, such as a seed-planting implement, a fertilizer-dispensing implement, and/or the like. Similarly, the work vehicle 12 may be configured as any other suitable type of vehicle, such as an agricultural harvester, a self-propelled sprayer, and/or the like.

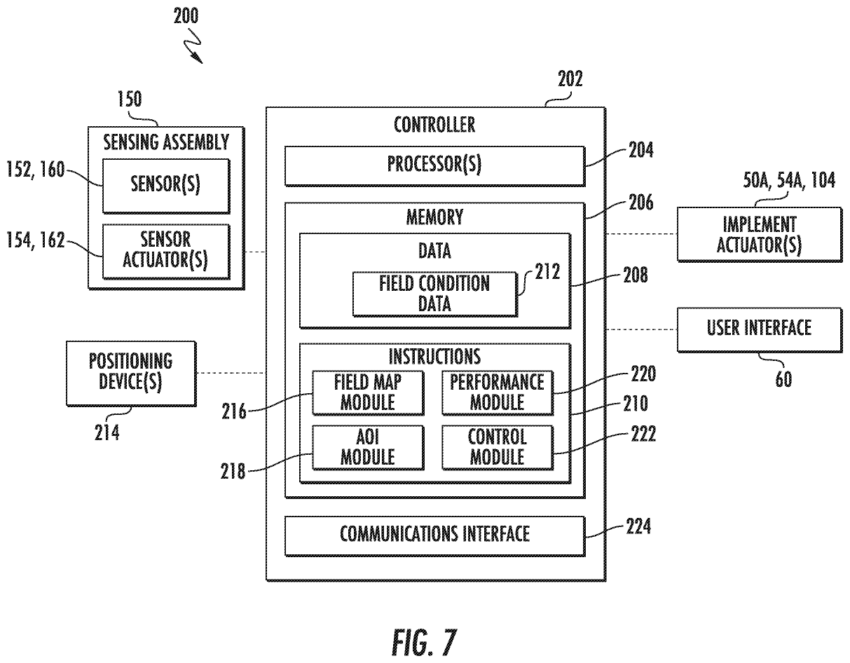

As shown in FIG. 1, the work vehicle 12 may include a pair of front track assemblies 16 (only one of which is shown) positioned at a front end 13 of the work vehicle 12, a pair of rear track assemblies 18 (only one of which is shown) positioned at a rear end 15 of the work vehicle 12, and a frame or chassis 20 coupled to and supported by the track assemblies 16, 18. An operator's cab 22 may be supported by a portion of the chassis 20 and may house various input devices (e.g., a user interface 60 shown in FIG. 7) for permitting an operator to control the operation of one or more components of the work vehicle 12 and/or one or more components of the implement 10. Additionally, the work vehicle 12 may include an engine 24 and a transmission 26 mounted on the chassis 20. The transmission 26 may be operably coupled to the engine 24 and may provide variably adjusted gear ratios for transferring engine power to the track assemblies 16, 18 via a drive axle assembly (not shown) (or via axles if multiple drive axles are employed).

As shown in FIGS. 1 and 2, the implement 10 may include a frame 28. More specifically, as shown in FIG. 2, the frame 28 may extend longitudinally between a forward end 30 and an aft end 32. The frame 28 may also extend laterally between a first side 34 and a second side 36. In this respect, the frame 28 generally includes a plurality of structural frame members 38, such as beams, bars, and/or the like, configured to support or couple to a plurality of components. Furthermore, a hitch assembly 40 may be connected to the frame 28 and configured to couple the implement 10 to the work vehicle 12. Additionally, a plurality of wheels 42 (one is shown) may be coupled to the frame 28 to facilitate towing the implement 10 in the direction of travel 14.

In several embodiments, the frame 28 may be configured to support one or more gangs or sets 44 of disc blades 46. Each disc blade 46 may, in turn, be configured to penetrate into or otherwise engage the soil as the implement 10 is being pulled through the field. In this regard, the various disc gangs 44 may be oriented at an angle relative to the direction of travel 14 to promote more effective tilling of the soil. In the embodiment shown in FIGS. 1 and 2, the implement 10 includes four disc gangs 44 supported on the frame 28 adjacent to its forward end 30. However, it should be appreciated that, in alternative embodiments, the implement 10 may include any other suitable number of disc gangs 44, such as more or fewer than four disc gangs 44. Furthermore, in one embodiment, the disc gangs 44 may be mounted to the frame 28 at any other suitable location, such as adjacent to its aft end 32.

Moreover, in several embodiments, the implement 10 may include a plurality of disc gang actuators 104 (FIG. 2), with each actuator 104 being configured to move or otherwise adjust the orientation or position of one of the disc gangs 44 relative to the implement frame 28. For example, as shown in the illustrated embodiment, a first end of each actuator 104 (e.g., a rod 106 of the actuator 104) may be coupled to a support arm 48 of the corresponding disc gang 44, while a second end of each actuator 104 (e.g., the cylinder 108 of the actuator 104) may be coupled to the frame 28. The rod 106 of each actuator 104 may be configured to extend and/or retract relative to the corresponding cylinder 108 to adjust the angle of the corresponding disc gang 44 relative to a lateral centerline (not shown) of the frame 28 and/or the penetration depth of the associated disc blades 46. In the illustrated embodiment, each actuator 104 corresponds to a fluid-driven actuator, such as a hydraulic or pneumatic cylinder. However, it should be appreciated that each actuator 104 may correspond to any other suitable type of actuator, such as an electric linear actuator.

Additionally, as shown, in one embodiment, the implement frame 28 may be configured to support other ground engaging tools. For instance, in the illustrated embodiment, the frame 28 is configured to support a plurality of shanks 50 or tines (not shown) configured to rip or otherwise till the soil as the implement 10 is towed across the field. Furthermore, in the illustrated embodiment, the frame 28 is also configured to support a plurality of leveling blades 52 and rolling (or crumbler) basket assemblies 54. The implement 10 may further include shank frame actuator(s) 50A and/or basket assembly actuator(s) 54A configured to move or otherwise adjust the orientation or position of the shanks 50 and the basket assemblies 54, respectively, relative to the implement frame 28. It should be appreciated that, in other embodiments, any other suitable ground-engaging tools may be coupled to and supported by the implement frame 28, such as a plurality closing discs.

It should be appreciated that the configuration of the implement 10 and work vehicle 12 described above are provided only to place the present subject matter in an exemplary field of use. Thus, it should be appreciated that the present subject matter may be readily adaptable to any manner of implement or work vehicle configurations.

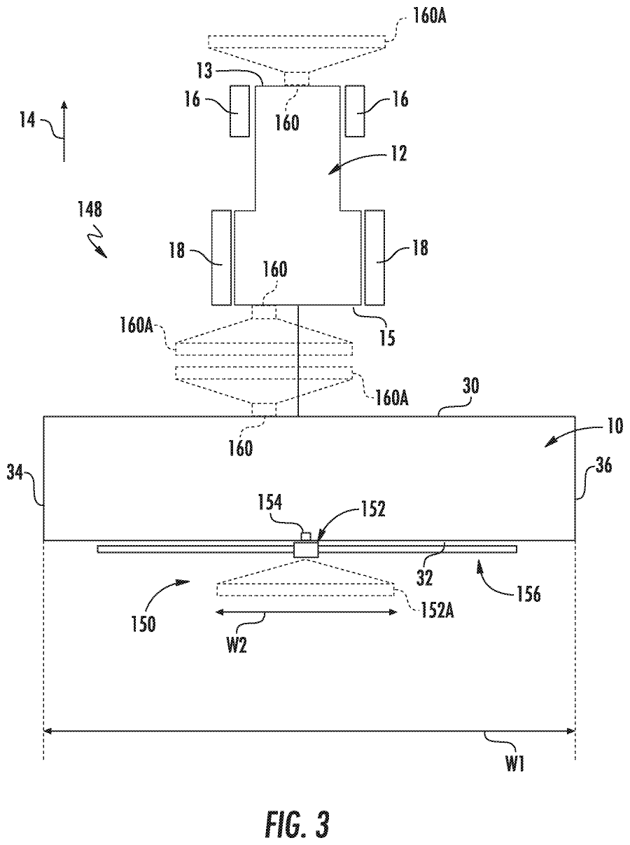

Referring now to FIG. 3, a schematic, top-down view of a system 148 provided in operative association with the implement 10 and the work vehicle 12 for monitoring field conditions as the implement 10 is moved across the field is illustrated in accordance with aspects of the present subject matter. As shown in FIG. 3, the system 148 may include a sensing assembly 150. The sensing assembly 150 may generally include a rearward sensor 152 supported on the implement 10, with the rearward sensor 152 having a field of view 152A directed towards the field. As shown in FIG. 3, in several embodiments, the rearward sensor 152 may be supported on and/or relative to the implement 10 by a support arm 156. It should be appreciated that the support arm 156 may be one of the frame members 38, 48 of the implement 10 described above, or may be a separate member coupled to the frame 28 of the implement 10.

In one embodiment, the rearward sensor 152 may be supported relative to the implement 10 such that the field of view 152A of the rearward sensor 152 is directed towards an aft portion of the field disposed rearward of the implement 10 relative to the direction of travel 14. For example, in the embodiment shown, the support arm 156 is positioned at or adjacent to the aft end 32 of the implement 10. As such, the rearward sensor 152 may be configured to generate data indicative of one or more field conditions associated with the aft portion of the field located behind or aft of the implement 10. For instance, the rearward sensor 152 may be configured to generate data indicative of at least one of a surface roughness, clod size, residue coverage, soil compaction, and/or the like of the aft portion of the field. The rearward sensor 152 may be configured as any suitable device, such as a camera(s) (including stereo camera(s), and/or the like), radar sensor(s), ultrasonic sensor(s), LIDAR device(s), infrared sensor(s), and/or the like such that the rearward sensor 152 generates image data, radar data, point-cloud data, infrared data, ultrasound data, and/or the like indicative of one or more monitored field conditions. For instance, the rearward sensor 152 may be configured as a radar sensor(s), an ultrasonic sensor(s), a LIDAR device(s), and/or a camera(s) to generate data indicative of soil roughness. Similarly, the rearward sensor 152 may be configured as a LIDAR device(s) and/or a camera(s) to generate data indicative of clod size and/or residue coverage. Further, the rearward sensor 152 may be configured as a radar sensor(s), specifically as ground-penetrating radar sensor(s), to generate data indicative of soil compaction.

In one embodiment, the field of view 152A of the rearward sensor 152 may be narrower than the implement 10 such that the rearward sensor 152 is only configured to capture data associated with a sub-section of the portion of the field located aft or behind the implement 10. More particularly, as shown in FIG. 3, the implement 10 has a width W1 extending between its first and second lateral sides 34, 36, which generally corresponds to the width of a swath of the field across which the implement 10 is configured to work the soil during the performance of the associated agricultural operation. In contrast, the field of view 152A of the rearward sensor 152 has a width W2 that is less than the width W1 of the implement 10 or worked field swath. For instance, in the embodiment shown, the width W2 of the field of view 152A corresponds to about one third of the width W1 of the implement/swath. However, it should be appreciated that, in other embodiments, the width W2 of the field of view 152A may correspond to any other suitable portion of the width W1 of the implement/swath, such as, for example, a quarter of the width W1, a half of the width W1, and/or the like. Thus, as the implement 10 is moved across the field, the sensor 152 is only configured to capture data associated with a portion of the field spanning across the width W1 of the implement 10.

Accordingly, as will be described in greater detail below, the disclosed sensing assembly 150 may also include an actuator 154 provided in operative association with the rearward sensor 152 that is configured to actuate the rearward sensor 152 relative to the implement 10 back and forth along a given sensor movement path such that the field of view 152A of the rearward sensor 152 can be oscillated across all or a given portion of the width W1 of the implement/swath, thereby allowing data to be captured along different sub-sections of the field swath being worked.

It should be appreciated that, while the sensing assembly 150 is shown as having only one rearward sensor 152, the sensing assembly 150 may have any other suitable number of rearward sensors 152, such as two or more rearward sensors 152. Further, while only one sensing assembly 150 is shown, the system 148 may have any other suitable number of sensing assemblies 150. Furthermore, in alternative embodiments, the sensing assembly 150 may be supported at any other suitable location on the implement 10 and/or the towing vehicle 12 such that the field of view 152A of the rearward sensor 152 is directed towards any other suitable portion of the field. For instance, in one embodiment, the sensing assembly 150 may be supported adjacent the forward end of the implement 10 or the aft end of the vehicle 12 such that the field of view 152A of the rearward sensor 152 is directed towards a portion of the field positioned immediately forward of the implement 10 (or immediately behind the vehicle 12) relative to the direction of travel 14. In another embodiment, the sensing assembly 150 may be supported adjacent the forward end of the vehicle 12 such that the field of view 152A of the rearward sensor 152 is directed towards a portion of the field positioned immediately forward of the vehicle 12 relative to the direction of travel 14.

Additionally, in some embodiments, the system 148 may include one or more forward sensors 160 configured to generate data indicative of one or more field conditions associated with a portion of the field prior to such field portions being worked by the implement 10. For instance, the forward sensor(s) 160 may be positioned at any suitable location relative to the implement 10 and/or work vehicle 12 such that a field of view 160A of each forward sensor 160 is directed towards a portion of the field disposed in front of the implement 10 and/or work vehicle 12 relative to the direction of travel 14. For example, the forward sensor(s) 160 may be positioned at a forward end 30 of the implement 10, at a rear end 15 of the work vehicle 12, or at a front end 13 of the work vehicle 12 as shown in FIG. 3. Accordingly, the forward sensor(s) 160 may generate data associated with initial surface roughness, clod sizes, residue coverage, soil compaction, and/or the like within the portion of the field. In other embodiments, the forward sensor(s) 160 may be configured to detect other field conditions, such as moisture content, and/or the like. It should be appreciated that the forward sensor(s) 160 may be configured as any suitable device, such as a camera(s) (including stereo camera(s), and/or the like), radar sensor(s), LIDAR device(s), infrared sensor(s), and/or the like.

In one embodiment, the forward sensor(s) may have a fixed field of view 160A relative to the portion of the associated implement 10 or work vehicle 12. However, in other embodiments, the forward sensor(s) 160 may be configured to be a part of a sensing assembly, similar to the rearward sensor 152 of the sensing assembly 150 described above, such that the forward sensor(s) 160 may be configured to be actuated back and forth along a sensor movement path relative to the portion of the associated implement 10 or work vehicle 12 by an actuator 162 (FIG. 7). As such, the field of view 160A of the forward sensor(s) 160 may be oscillated across all or a given portion of the width W of the implement/swath.

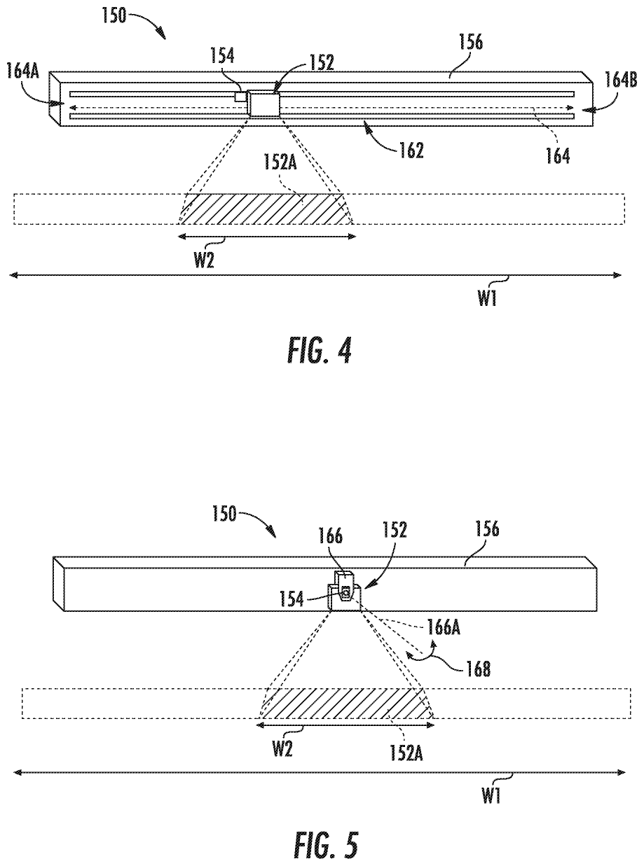

Referring now to FIGS. 4 and 5, exemplary embodiments of sensor movement paths along which the rearward sensor(s) 152 of the disclosed sensing assembly 150 may be actuated are illustrated in accordance with aspects of the present subject matter. More particularly, FIG. 4 illustrates a linear sensor movement path along which the rearward sensor(s) 152 may be actuated. Additionally, FIG. 5 illustrates an arced or curved sensor movement path along which the rearward sensor(s) 152 may be actuated.

As shown in FIG. 4, in several embodiments, the rearward sensor 152 may be supported on the implement 10 (e.g., via the support arm 156) such that the rearward sensor 152 is linearly actuatable relative to the support arm 156 and/or the adjacent portion of the implement 10. More particularly, the rearward sensor 152 may be configured to be actuated by the associated actuator 154 relative to the support arm 156 and/or the adjacent portion of the implement 10 along a substantially linear movement path 164 extending between a first end 164A and a second end 164B. As indicated above, the actuator 154 may be configured to move the rearward sensor 152 back and forth along the linear movement path 164 as the implement 10 is moved across the field such that a field of view 152A of the rearward sensor 152 is oscillated across the width W1 of the implement/swath, allowing data to be captured along different sub-sections of the field swath being worked.

The actuator 154 may correspond to any suitable actuation device that is configured to drive the rearward sensor 152 along the linear movement path 164. For instance, in a particular embodiment, the rearward sensor 152 is coupled to the support arm 156 by a rail system 162. One or more of the rails of the rail system 162 may be configured as a fixed rack configured to engage a corresponding pinion gear coupled to the actuator 154. In such an embodiment, the actuator 154 may correspond to a rotary actuator (e.g., an electric motor) configured to rotationally drive the pinion gear to linearly actuate the rearward sensor 152 along the linear movement path 164.

It should be appreciated that, in alternative embodiments, the rearward sensor 152 may be coupled to the support arm 156 by any other suitable means that allows the rearward sensor 152 to be actuated along the linear movement path 164. For instance, the rearward sensor 152 may be coupled to the support arm 156 by a track, a parallel linkage assembly, a pivoting arm, and/or the like. Furthermore, it should be appreciated that the actuator 154 may correspond to any suitable actuator that is configured to actuate the rearward sensor 152 along an associated linear movement path 164. For instance, the actuator 154 may be configured as a hydraulic cylinder, a pneumatic cylinder, a belt drive, a screw drive, and/or the like.

As shown in FIG. 5, the rearward sensor 152 may alternatively be supported on the implement 10 such that the rearward sensor 152 is pivotably actuatable relative to the support arm 156 and/or the adjacent portion of the implement 10. For example, the rearward sensor 152 may be coupled to the support arm 156 by a pivot bracket 166 such that the rearward sensor 152 is pivotable about a horizontal pivot axis 166A along an arced movement path 168 corresponding to a range of angular positions of the rearward sensor 152. In such an embodiment, the actuator 154 may be configured to move the rearward sensor 152 back and forth along the arced movement path 168 as the implement 10 is moved across the field such that a field of view 152A of the rearward sensor 152 is oscillated across the width W1 of the implement/swath, allowing data to be captured along different sub-sections of the field swath being worked. For instance, in the embodiment shown, the actuator 154 is a rotary actuator mounted to the pivot bracket 166 and configured to rotate the rearward sensor 152 along the arced movement path 168. It should be appreciated that, in alternative embodiments, the rearward sensor 152 may be coupled to the support arm 156 by any other suitable means that allows the rearward sensor 152 to be pivotably actuated along the arced movement path 168. For instance, the rearward sensor 152 may be coupled to the support arm 156 by a rack-and-pinion system, a worm assembly, and/or the like. Furthermore, it should be appreciated that the actuator 154 may correspond to any suitable actuator configured to actuate the rearward sensor 152 along the arced movement path 168. For instance, the actuator 154 may be configured as a hydraulic cylinder, a pneumatic cylinder, a belt drive, a worm gear drive, and/or the like.

FIGS. 4 and 5 illustrate differing configurations for actuating the rearward sensor 152 across a linear movement path and an arced movement path, respectively. However, it should be appreciated that, in other embodiments, the sensing assembly 150 may include an actuator, or a combination of actuators, configured to both linearly and pivotably actuate the rearward sensor 152 such that the rearward sensor 152 is movable along both a linear movement path and an arced movement path.

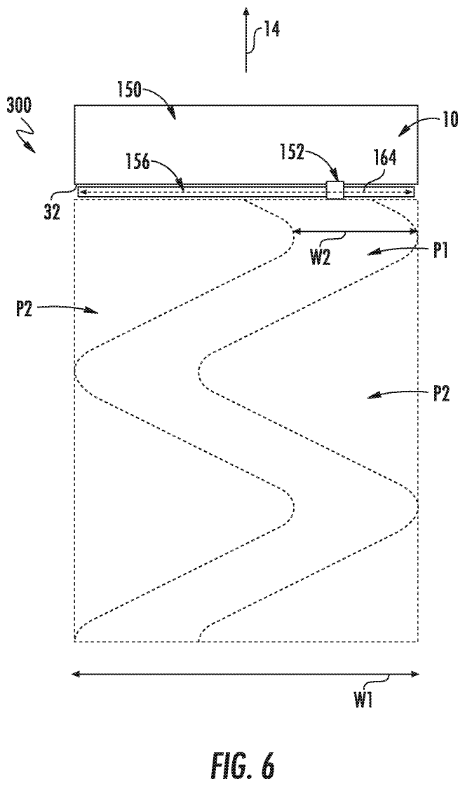

Referring now to FIG. 6, an example view of an aft end of the implement and an adjacent portion of a field are illustrated in accordance with aspects of the present subject matter. More particularly, FIG. 6 shows a portion 300 of a field adjacent to an aft end of the implement during operation of the sensing assembly 150 in which the rearward sensor 152 is configured to be actuated back and forth along the sensor movement path (e.g., the linear movement path 164) such that its field of view 152A is oscillated back and forth along the width W1 of the implement/swath while the implement 10 is moved across the field. In some embodiments, the rearward sensor 152 is continuously actuated back and forth along the linear sensor movement path 164 at a relatively constant speed. As such, the field of view 152A of the rearward sensor 152 may generally follow a sinusoidal path such that the rearward sensor 152 collects data corresponding to a sine-shaped first sub-portion P1 of the swath. However, in other embodiments, the rearward sensor 152 may be actuated such that its field of view 152A follows any other shaped path. Further, in some embodiments, such as the embodiment shown, the rearward sensor 152 is actuated across the linear movement path 164 such that its field of view 152A is oscillated across the entire width W1 of the implement/swath. It should be appreciated, however, that the rearward sensor 152 may be oscillated to cover any suitable portion of the width W1 of the implement/swath.

The data generated by the rearward sensor 152 as the implement 10 is moved across the field may be used to generate a field condition map. As indicated above, in certain embodiments, the rearward sensor 152 generates data indicative of a field condition(s) for only a portion of the field due to its oscillating field of view as the sensor 152 is actuated back and forth along its sensor movement path, such as the first sub-portion(s) P1 of the field shown in FIG. 6. In such embodiments, to determine the field condition(s) for the remaining portions of the field, it may be assumed that the portions of the field outside of the sensor's field of view (e.g., second sub-portions P2 shown in FIG. 6) have the same or similar field condition(s) as the first sub-portions P1 of the swath for each position of the implement 10 within the field. As such, a field map may be generated that correlates a field condition(s) to each position within the field based on the data generated by the rearward sensor 152. The field map may generally be used to control the operation of an implement performing a subsequent agricultural operation.

Referring now to FIG. 7, a schematic view of another embodiment of a system 200 for monitoring field conditions as an agricultural implement is moved across a field is illustrated in accordance with aspects of the present subject matter. In general, the system 200 will be described herein with reference to the implement 10 and the work vehicle 12 described above with reference to FIGS. 1-3, as well as the system 148 described above with reference to FIGS. 3-6. However, it should be appreciated by those of ordinary skill in the art that the disclosed system 200 may generally be utilized with work vehicles having any suitable vehicle configuration, implements having any suitable implement configuration, and/or with sensing assemblies having any other suitable assembly configuration. Additionally, it should be appreciated that, for purposes of illustration, communicative links or electrical couplings of the system 200 shown in FIG. 7 are indicated by dashed lines.

In several embodiments, the system 200 may include a controller 202 and various other components configured to be communicatively coupled to and/or controlled by the controller 202, such as a sensing assembly (e.g., sensing assembly 150) having one or more sensors configured to capture field conditions of a field (e.g., sensor(s) 152,160) and one or more actuators (e.g., actuator(s) 154, 162), a user interface (e.g., user interface 60), various components of the implement 10 and/or the work vehicle 12 (e.g., implement actuator(s) 50A, 54A, 104), and/or various other components of the sensing assembly 150 (e.g., actuator(s) 154, 162). The user interface 60 described herein may include, without limitation, any combination of input and/or output devices that allow an operator to provide operator inputs to the controller 202 and/or that allow the controller 202 to provide feedback to the operator, such as a keyboard, keypad, pointing device, buttons, knobs, touch sensitive screen, mobile device, audio input device, audio output device, and/or the like.

In general, the controller 202 may correspond to any suitable processor-based device(s), such as a computing device or any combination of computing devices. Thus, as shown in FIG. 7, the controller 202 may generally include one or more processor(s) 204 and associated memory devices 206 configured to perform a variety of computer-implemented functions (e.g., performing the methods, steps, algorithms, calculations and the like disclosed herein). As used herein, the term “processor” refers not only to integrated circuits referred to in the art as being included in a computer, but also refers to a controller, a microcontroller, a microcomputer, a programmable logic controller (PLC), an application specific integrated circuit, and other programmable circuits. Additionally, the memory 206 may generally comprise memory element(s) including, but not limited to, computer readable medium (e.g., random access memory (RAM)), computer readable non-volatile medium (e.g., a flash memory), a floppy disk, a compact disc-read only memory (CD-ROM), a magneto-optical disk (MOD), a digital versatile disc (DVD) and/or other suitable memory elements. Such memory 206 may generally be configured to store information accessible to the processor(s) 204, including data 208 that can be retrieved, manipulated, created and/or stored by the processor(s) 204 and instructions 210 that can be executed by the processor(s) 204.

It should be appreciated that the controller 202 may correspond to an existing controller for the implement 10 or the vehicle 12 or may correspond to a separate processing device. For instance, in one embodiment, the controller 202 may form all or part of a separate plug-in module that may be installed in operative association with the implement 10 or the vehicle 12 to allow for the disclosed system and method to be implemented without requiring additional software to be uploaded onto existing control devices of the implement 10 or the vehicle 12.

In several embodiments, the data 208 may be stored in one or more databases. For example, the memory 206 may include a field condition database 212 for storing field condition data received from the sensor(s) 152, 160. For instance, the sensor(s) 152, 160 may be configured to continuously or periodically capture data associated with a portion of the field, such as immediately before and/or after the performance of an agricultural operation within such portion of the field. In such an embodiment, the data transmitted to the controller 202 from the sensor(s) 152, 160 may be stored within the field condition database 212 for subsequent processing and/or analysis. It should be appreciated that, as used herein, the term field condition data 212 may include any suitable type of data received from the sensor(s) 152, 160 that allows for the field conditions of a field to be analyzed, including photographs or other images, RADAR data, LIDAR data, and/or other image-related data (e.g., scan data and/or the like).

It should be appreciated that, in several embodiments, the field condition data 212 may be geo-referenced or may otherwise be stored with corresponding location data associated with the specific location at which such data was collected within the field. In one embodiment, the field condition data 212 may be correlated to a corresponding position within the field based on location data received from one or more positioning devices. For instance, the controller 202 may be communicatively coupled to a positioning device(s) 214, such as a Global Positioning System (GPS) or another similar positioning device, configured to transmit a location corresponding to a position of the sensor(s) 152, 160 within the field when field condition data 212 is collected by the sensor(s) 152, 160.

Referring still to FIG. 7, in several embodiments, the instructions 210 stored within the memory 206 of the controller 202 may be executed by the processor(s) 204 to implement a field map module 216. In general, the field map module 216 may be configured to analyze the field condition data 212 deriving from the sensor(s) 152, 160 to generate a field condition map for the field. For instance, as described above, the field condition data 212 detected by the sensor(s) 152, 260 may correspond to a parameter indicative of a field condition at a given position within the field, e.g., the field condition of first sub-portions P1 (FIG. 6) of a swath for each position within the field. The field map module 216 may generally correlate the parameter indicative of the field condition to the actual field condition (e.g., surface roughness, clod size, crop residue coverage, soil compaction) at each position. The field map module 216 may then, for example, be configured to generate a field condition map based on the assumption that other portions of the field, e.g., second sub-portions P2 (FIG. 6) of a swath outside of or adjacent to the first sub-portions P1 for each position within the field, have the same field conditions as the first sub-portions P1.

Further, in some embodiments, the instructions 210 stored within the memory 206 of the controller 202 may be executed by the processor(s) 204 to implement an area-of-interest (AOI) module 218. In one embodiment, the AOI module 218 may be configured to automatically analyze the field condition data 212 deriving from the sensor(s) 152, 160 to determine an area-of-interest. For instance, the AOI module 218 may compare the data from the sensor(s) 152, 160 to one or more associated thresholds and determine an area-of-interest within the field when the data crosses such threshold(s). For example, the AOI module 218 may monitor the surface roughness, clod size, residue coverage, and/or soil compaction of the field from data received from the sensor(s) 152, 160 and determine an area-of-interest when the surface roughness, clod size, residue coverage, and/or soil compaction exceeds and/or drops below an associated threshold. In other embodiments, the AOI module 218 may similarly monitor the data from the forward sensor(s) 160 to determine an area-of-interest when the data crosses such threshold(s). In further embodiments, the AOI module 218 may monitor data from one or more auxiliary sensors (not shown) indicative of the vibrations or levelness of the implement 10 and/or the moisture content of the field and determine an area-of-interest when the vibrations, levelness, or moisture content exceeds and/or drops below an associated threshold. In additional embodiments, the controller 202 may receive an indication of such area-of-interest from an operator, e.g., via the user interface 60.

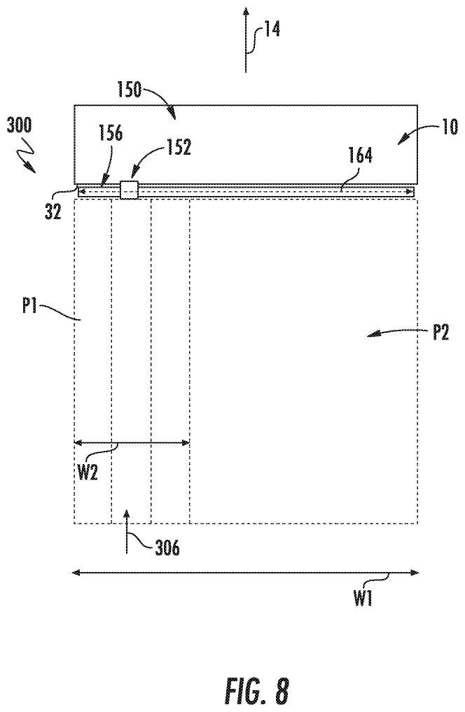

Referring briefly to FIG. 8, a portion 300 of a field adjacent to an aft end of the implement is illustrated following the identification of an area-of-interest 306 within the field. In particular, upon determining the location of the area-of-interest 306, the rearward sensor 152 is configured to be actuated along the sensor movement path (e.g., the linear movement path 164) such that its field of view 152A is directed towards the area-of interest 306. In some embodiments, the rearward sensor 152 is configured to remain static while monitoring the area-of-interest 306. However, in other embodiments, the rearward sensor 152 may be actuated back and forth along the linear sensor movement path 164 such that the field of view 152A of the rearward sensor 152 may oscillate while at least partially maintaining the area-of-interest 306 within the field of view 152A. In general, the rearward sensor 152 generates data corresponding to a first sub-portion P1 of the swath, including the area-of-interest 306. The AOI module 218 may further be configured to monitor the data from the sensor indicative of the field conditions within the area-of-interest to determine whether the implement 10 is performing properly across the swath width W1. Particularly, it can be determined whether the settings of the implement 10 are correct for the field conditions, such that the field is being worked properly, or if there is a problem with the implement 10, such as with the leveling of the implement 10 or plugging of the tools. The operation of the implement 10, specifically the operation of one or more components of the implement 10, may be adjusted based on the determined field conditions to improve the field conditions during the working of the field by the implement 10.

Referring back to FIG. 7, in some embodiments, the instructions 210 stored within the memory 206 of the controller 202 may be executed by the processor(s) 204 to implement a performance module 220. In general, the performance module 220 may be configured to compare the field condition data 212 deriving from the sensor(s) 152, 160 to determine a performance of the implement 10. For instance, as indicated above, in one embodiment, data may be captured for the same section of the field by the forward sensor(s) 160 before the agricultural operation has been performed and by the rearward sensor 152 after the agricultural operation has been performed. In such an embodiment, the performance module 220 may be configured to analyze the pre-operation and post-operation data to determine a field condition differential for the analyzed section of the field, which can then be used to assess the performance of the implement 10. For instance, data from the forward sensor(s) 160 may be used to detect the soil roughness of the portion of the field immediately in front of the vehicle 12 and/or implement 10 prior to working such portion of the field and the data from the rearward sensor(s) 152 may be configured to detect the soil roughness of the same portion of the field immediately behind the implement 10 following the performance of the agricultural operation. The pre-operation soil roughness may then be compared to the post-operation soil roughness to assess the effectiveness of the implement 10 in performing the operation.

Additionally, in some embodiments, the instructions 210 stored within the memory 206 of the controller 202 may be executed by the processor(s) 204 to implement a control module 222. In some embodiments, the control module 222 may be configured to adjust a position of one or more components of the implement 10, the sensing assembly 150, and/or the user interface 60 based on the monitored field conditions. For instance, in some embodiments, the control module 222 may be configured to adjust the downforce acting on components of the implement 10 by one or more of the actuators 50A, 54A, 104 to improve the field surface conditions based on the monitored field conditions and/or performance of the implement 10. In some embodiments, the control module 222 may control the actuation of the actuator 154 to move the sensor 152 such that the field of view 152A of the sensor 152 is directed towards the area-of-interest determined by the AOI module 218 for monitoring the field condition(s) of the area-of-interest. In some embodiments, the control module 222 may be configured to adjust the operation of the implement 10 based on an input from the operation, e.g., via the user interface 60. Additionally or alternatively, in some embodiments, the controller 202 may further be configured to control the operation of the user interface 60 to notify an operator of the field conditions, performance efficiency of the implement 10, and/or the like.

Moreover, as shown in FIG. 7, the controller 202 may also include a communications interface 224 to provide a means for the controller 202 to communicate with any of the various other system components described herein. For instance, one or more communicative links or interfaces (e.g., one or more data buses) may be provided between the communications interface 224 and the sensor(s) 152, 160 to allow data transmitted from the sensor(s) 152, 160 to be received by the controller 202. Similarly, one or more communicative links or interfaces (e.g., one or more data buses) may be provided between the communications interface 224 and the user interface 60 to allow operator inputs to be received by the controller 202 and to allow the controller 202 to control the operation of one or more components of the user interface 60 to present field conditions to the operator.

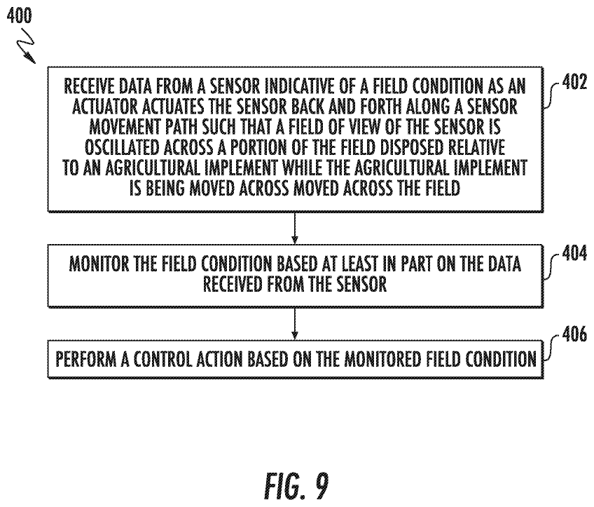

Referring now to FIG. 9, a flow diagram of one embodiment of a method 400 for monitoring field conditions as an agricultural operation is performed within a field is illustrated in accordance with aspects of the present subject matter. In general, the method 400 will be described herein with reference to the implement 10 and the work vehicle 12 shown in FIGS. 1-3, as well as the sensing assembly 150 shown in FIGS. 3-6 and the various system components shown in FIG. 7. However, it should be appreciated that the disclosed method 400 may be implemented with work vehicles and/or implements having any other suitable configurations, with sensing assemblies having any other suitable configurations, and/or within systems having any other suitable system configuration. In addition, although FIG. 9 depicts steps performed in a particular order for purposes of illustration and discussion, the methods discussed herein are not limited to any particular order or arrangement. One skilled in the art, using the disclosures provided herein, will appreciate that various steps of the methods disclosed herein can be omitted, rearranged, combined, and/or adapted in various ways without deviating from the scope of the present disclosure.

As shown in FIG. 9, at (402), the method 400 may include receiving data from a sensor indicative of a field condition as an actuator actuates the sensor back and forth along a sensor movement path such that a field of view of the sensor is oscillated across a portion of the field disposed relative to an agricultural implement while the agricultural implement is being moved across the field. For instance, as described above, the controller 202 may be configured to receive data from the sensor 152 as it is actuated back and forth along the sensor movement path 164, 168 such that the field of view 152A of the sensor 152 is oscillated across a portion of the field disposed forward or rearward of the implement 10 while the implement 10 is being moved across the field (e.g., in the direction of travel 14).

Further, at (404), the method 400 may include monitoring the field condition based at least in part on the data received from the sensor. For example, as described above, the controller 202 may monitor one or more field conditions associated with the portions of the field captured within the field of view of the sensor based on an assessment or analysis of the data received from the sensor 152. For instance, based on the type of sensor being used and/or the type of data being collected, the controller 202 may be configured to monitor the soil roughness within the field, clod sizes, crop residue coverage, soil compaction, and/or the like.

Additionally, at (406), the method 400 may include performing a control action based on the monitored field condition. For instance, as described above, the control action may include automatically controlling one or more components of the implement 10 (e.g., by controlling one or more of the actuators 50A, 54A, 104) to adjust the operation of the implement 10 in a manner that varies the monitored field condition, controlling the operation of the sensor actuator 164 to move the sensor 152 to adjust the field of view 152A of the sensor 152 (e.g., direct the field of view 152A towards an area-of-interest), and/or notifying an operator of the present field conditions.

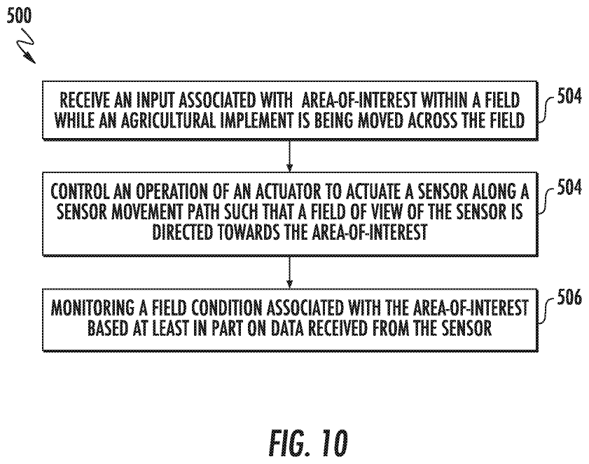

Referring now to FIG. 10, a flow diagram of another embodiment of a method 500 for monitoring field conditions as an agricultural operation is performed within a field is illustrated in accordance with aspects of the present subject matter. In general, the method 500 will be described herein with reference to the implement 10 and the work vehicle 12 shown in FIGS. 1-3, as well as the sensing assembly 150 shown in FIGS. 3-6 and the various system components shown in FIG. 7. However, it should be appreciated that the disclosed method 500 may be implemented with work vehicles and/or implements having any other suitable configurations, with sensing assemblies having any other suitable configurations, and/or within systems having any other suitable system configuration. In addition, although FIG. 10 depicts steps performed in a particular order for purposes of illustration and discussion, the methods discussed herein are not limited to any particular order or arrangement. One skilled in the art, using the disclosures provided herein, will appreciate that various steps of the methods disclosed herein can be omitted, rearranged, combined, and/or adapted in various ways without deviating from the scope of the present disclosure.

As shown in FIG. 10, at (502), the method 500 may include receiving an input associated with an area-of-interest within a field while an agricultural implement is being moved across the field. For instance, as described above, the controller 202 may be configured to receive an input from one or more sensors 152, 160 or an operator, e.g., via the user interface 60, indicative of an area-of-interest while the implement 10 is moved across the field. The controller 202 may further be configured to determine a specific area-of-interest by analyzing the data received from the sensor(s) 152, 160 (e.g., by comparing the data received from the sensor(s) 152, 160 to one or more thresholds and determining an area-of-interest when the data exceeds or falls below an associated threshold) or may automatically determine the area-of-interest upon receipt of an input from the operator.

Further, at (504), the method 500 may include controlling an operation of an actuator to actuate a sensor along a sensor movement path such that a field of view of the sensor is directed towards the area-of-interest. As indicated above, the controller 202 may be configured to control the operation of the actuator 154 to actuate the rearward sensor 152 such that the field of view 152A of the rearward sensor 152 is directed towards the area-of-interest 306, where the rearward sensor 152 generates data indicative of the field conditions within the area-of-interest 306 while the implement 10 continues to move across the field.

Additionally, at (506), the method 500 may include monitoring a field condition associated with the area-of-interest based at least in part on data received from the sensor. As described above, the controller 202 may be configured to monitor the data received from the rearward sensor 152 associated with a field condition(s) within the area-of-interest to determine a field condition within the area-of-interest.

It is to be understood that, in several embodiments, the steps of the methods 400, 500 are performed by the controller 202 upon loading and executing software code or instructions which are tangibly stored on a tangible computer readable medium, such as on a magnetic medium, e.g., a computer hard drive, an optical medium, e.g., an optical disc, solid-state memory, e.g., flash memory, or other storage media known in the art. Thus, in several embodiments, any of the functionality performed by the controller 202 described herein, such as the methods 400, 500, are implemented in software code or instructions which are tangibly stored on a tangible computer readable medium. The controller 202 loads the software code or instructions via a direct interface with the computer readable medium or via a wired and/or wireless network. Upon loading and executing such software code or instructions by the controller 202, the controller 202 may perform any of the functionality of the controller 202 described herein, including any steps of the methods 400, 500 described herein.

The term “software code” or “code” used herein refers to any instructions or set of instructions that influence the operation of a computer or controller. They may exist in a computer-executable form, such as machine code, which is the set of instructions and data directly executed by a computer's central processing unit or by a controller, a human-understandable form, such as source code, which may be compiled in order to be executed by a computer's central processing unit or by a controller, or an intermediate form, such as object code, which is produced by a compiler. As used herein, the term “software code” or “code” also includes any human-understandable computer instructions or set of instructions, e.g., a script, that may be executed on the fly with the aid of an interpreter executed by a computer's central processing unit or by a controller.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they include structural elements that do not differ from the literal language of the claims, or if they include equivalent structural elements with insubstantial differences from the literal languages of the claims.

What is claimed is:

1. A system for monitoring field conditions of a field, the system comprising:
a sensor supported on an agricultural implement such that the sensor has a field of view directed towards an aft portion of the field disposed rearward of the agricultural implement relative to a direction of travel of the agricultural implement, the sensor being configured to generate data indicative of a field condition associated with the aft portion of the field;
an actuator configured to actuate the sensor back and forth relative to an adjacent portion of the agricultural implement along a sensor movement path; and
a controller configured to:
receive data from the sensor indicative of the field condition as the actuator actuates the sensor back and forth along the sensor movement path such that the field of view of the sensor is oscillated across the aft portion of the field while the agricultural implement is being moved across the field; and
monitor the field condition based at least in part on the data received from the sensor.

2. The system of claim 1, wherein the actuator is configured to linearly actuate the sensor such that the sensor movement path comprises a linear movement path relative to the adjacent portion of the agricultural implement.

3. The system of claim 1, wherein the actuator is configured to pivotably actuate the sensor such that the sensor movement path comprises an arced movement path relative to the adjacent portion of the agricultural implement.

4. The system of claim 1, wherein the data generated by the sensor is associated with the field condition along a first sub-section of the aft portion of the field across which the field of view of the sensor is oscillated, the controller being further configured to estimate the field condition associated with a second sub-section of the aft portion of the field outside of the field of view of the sensor based at least in part on the data associated with the first sub-section of the aft portion of the field.

5. The system of claim 4, wherein the controller is further configured to generate a field map correlating the field condition to the first and second sub-sections of the aft portion of the field.

6. The system of claim 1, wherein the controller is further configured to:
determine an area-of-interest within the field based at least in part on the data received from the sensor, and
control the actuator such that a field of view of the sensor is directed towards the area-of-interest.

7. The system of claim 1, further comprising a second sensor supported on the agricultural implement such that the second sensor has a field of view directed towards a forward portion of the field disposed in front of the agricultural implement relative to the direction of travel of the agricultural implement, the second sensor being configured to generate data indicative of the field condition for the forward portion of the field, the controller being configured to compare the data associated with the monitored field condition for the forward and aft portions of the field to assess the effectiveness of an agricultural operation being performed in the field with the agricultural implement.

8. The system of claim 1, wherein the field condition comprises at least one of a surface roughness, clod size, residue coverage, or soil compaction.

9. The system of claim 1, wherein the controller is further configured to perform a control action based at least in part on the monitored field condition.

10. A system for monitoring field conditions of a field, the system comprising:
a sensor supported on an agricultural implement such that the sensor has a field of view directed towards the field, the sensor being configured to generate data indicative of a field condition associated with the field;
an actuator configured to actuate the sensor back and forth relative to an adjacent portion of the agricultural implement along a sensor movement path; and
a controller configured to:
determine an area-of-interest within the field;
control an operation of the actuator to actuate the sensor along the sensor movement path such that the field of view is directed towards the area-of-interest within the field; and
monitor the field condition associated with the area-of-interest based at least in part on the data received from the sensor.

11. The system of claim 10, wherein the controller is configured to determine the area-of-interest within the field based at least in part on at least one of sensor data received from the sensor, sensor data received from a secondary sensor, or an input received from an operator of the agricultural implement.

12. The system of claim 11, wherein the sensor is configured to generate data indicative of the field condition associated with an aft portion of the field relative to the agricultural implement in a direction of travel of the agricultural implement and the secondary sensor is configured to generate data indicative of the field condition associated with a forward portion of the field relative to the agricultural implement in the direction of travel.

13. The system of claim 10, wherein the controller is further configured to adjust an operation of one or more components of the implement based on the monitored field condition within the area-of-interest.

14. A system for monitoring field conditions of a field, the system comprising:
a sensor supported on an agricultural implement such that the sensor has a field of view directed towards a portion of the field, the sensor being configured to generate data indicative of a field condition associated with the portion of the field:
an actuator configured to linearly actuate the sensor back and forth relative to an adjacent portion of the agricultural implement along a linear movement path; and
a controller configured to:
receive data from the sensor indicative of the field condition as the actuator linearly actuates the sensor back and forth along the linear movement path such that the field of view of the sensor is oscillated across the portion of the field while the agricultural implement is being moved across the field; and
monitor the field condition based at least in part on the data received from the sensor.

15. The system of claim 14, wherein the data generated by the sensor is associated with the field condition along a first sub-section of the portion of the field across which the field of view of the sensor is oscillated, the controller being further configured to estimate the field condition associated with a second sub-section of the portion of the field outside of the field of view of the sensor based at least in part on the data associated with the first sub-section of the portion of the field.

16. The system of claim 15, wherein the controller is further configured to generate a field map correlating the field condition to the first and second sub-sections of the portion of the field.

17. The system of claim 14, wherein the controller is further configured to:
determine an area-of-interest within the field based at least in part on the data received from the sensor, and
control the actuator such that a field of view of the sensor is directed towards the area-of-interest.

18. The system of claim 14, wherein the sensor is supported on the agricultural implement such that the field of view of the sensor is directed towards an aft portion of the field disposed rearward of the agricultural implement relative to a direction of travel of the agricultural implement.

19. The system of claim 18, further comprising a second sensor supported on the agricultural implement such that the second sensor has a field of view directed towards a forward portion of the field disposed in front of the agricultural implement relative to the direction of travel of the agricultural implement, the second sensor being configured to generate data indicative of the field condition for the forward portion of the field, the controller being configured to compare the data associated with the monitored field condition for forward and aft portions of the field to assess the effectiveness of an agricultural operation being performed in the field with the agricultural implement.

20. The system of claim 14, wherein the field condition comprises at least one of a surface roughness, clod size, residue coverage, or soil compaction.
